(19) United States
(12) Patent Application Publication
(10) Pub. No.: US 2026/0335627 A1
(43) Pub. Date: Oct. 29, 2026

(54) LEFT VENTRICULAR ASSIST DEVICE, COMPONENTS AND METHODS

(71) Applicants: David Allan Melander, St. Louis Park, MN (US); Joseph Wang, Henderson, NV (US)

(72) Inventors: David Allan Melander, St. Louis Park, MN (US); Joseph Wang, Henderson, NV (US)

(21) Appl. No.: 19/727,245

(22) Filed: Jul. 1, 2026

Related U.S. Application Data
(63) Continuation-in-part of application No. 19/011,198, filed on Jan. 6, 2025.

(51) Int. Cl. A61M 60/178; A61M 60/232; A61M 60/422; A61M 60/515; A61M 60/538; A61M 60/816; A61M 60/90

(52) U.S. Cl. CPC A61M 60/178; A61M 60/232; A61M 60/422; A61M 60/515; A61M 60/538; A61M 60/816; A61M 60/90; A61M 2205/04; A61M 2205/3317; A61M 2205/50; A61M 2230/005; A61M 2230/62

(57) ABSTRACT

Embodiments herein relate to a left ventricle assist device (LVAD) including an inflow port; an outflow port; an impeller configured to pump blood from the inflow port to the outflow port so as to assist the left ventricle of a heart in pumping blood; and a motor driving the impeller, the motor is included having a stator element; a magnetic disc; wherein the magnetic disc includes programmable multi-pole magnets. Other embodiments are also included herein.

### CROSS-REFERENCE TO RELATED APPLICATIONS

This application is a continuation-in-part of U.S. patent application Ser. No. 19/011,198, filed on Jan. 6, 2025, entitled LEFT VENTRICAL ASSIST DEVICE AND COMPONENTS AND METHODS; which claims priority to U.S. Provisional Patent Application No. 63/618,287, filed on Jan. 6, 2024, entitled LEFT VENTRICAL ASSIST DEVICE AND COMPONENTS AND METHODS. All of these applications are herein incorporated by reference in their entireties.

### TECHNICAL FIELD

Embodiments herein relate to left ventricle assist devices (LVADs), including components used in LVADs and methods of making and using LVADs.

### BACKGROUND

A left ventricular assist device (LVAD) is a pump typically used to treat patients who have reached end-stage heart failure. LVADs are typically battery-operated mechanical pumps that are typically implanted into a patient and help the left ventricle pump blood to the rest of the body. LVADs can be used as a temporary bridge to a heart transplant when recipients use the LVAD until a heart becomes available. LVADs can also be used to restore function in a failing heart, eliminating the need for a transplant, at least temporarily. This is especially useful because some patients are not candidates for heart transplants. In this case, patients can receive long-term treatment using an LVAD, which can prolong and improve their lives.

Heart failure is a complex clinical syndrome resulting from any structural or functional impairment of ventricular filling or ejection of blood and affects more than 6 million Americans. Advanced heart failure is defined as the presence of refractory symptoms despite optimal medical, surgical, and device therapy. It is estimated that approximately half a million individuals have late state heart failure in the United States, with millions more in other countries. Approximately 10 to 15 percent of these patients are eligible for advanced therapies yearly, but only a small percent receive an LVAD or heart transplant, leaving a large percentage of eligible patients deprived of these life-saving therapies. Also, despite efforts to increase the donor pool, heart transplant remains limited by the scarcity of donor hearts. However, LVAD therapy does not share the same constraints.

Although LVADs are useful and can save lives, improve health, and prolong lives, there remains a need for improved LVAD designs.

### SUMMARY

LVADs are often charactered as continuous-flow or pulsatile. Continuous-flow LVADs are smaller and have proven to be more durable than pulsatile LVADs. They normally use either a centrifugal pump or an axial flow pump. Both types have a central rotor containing permanent magnets. Controlled electric currents running through coils contained in the pump housing apply forces to the magnets, which in turn cause the rotor or rotors to spin. In the centrifugal pumps, the rotors are shaped to accelerate the blood circumferentially and thereby cause it to move toward the outer rim of the pump, whereas in the axial flow pumps the rotors are more or less cylindrical with blades that are helical, causing the blood to be accelerated in the direction of the rotor's axis.

The present application utilizes a permanent magnetic structure having a programmable multi-pole magnet (e.g., generically depicting the technology commercialized as a Polymagnet™) typically made of neodymium, that has been precisely arranged with a custom pattern of alternating magnetic polarities on a single piece of material. Unlike conventional magnets that possess only a single north and south pole, these permanent magnetic structures feature localized micro-poles arranged in specific geometric arrays. This engineered alignment concentrates the magnetic flux near the surface, allowing developers to program highly customized mechanical behaviors such as precise alignment guidance, zero-force tactile latching, shear-force resistance, and rapid, localized force cancellation as the structures are rotated or separated.

Benefits of LVADs made using programmable multi-pole magnets include reduction of mass by: (a) dramatic reduction of mass of the permanent rare earth; (b) reduction of size of windings, coils; and (c) the ability to make general reductions of all materials. In addition, there can be a reduction in power consumption, allowing greater battery life and reduction in size of external power management systems. Greater proximities of coils to specific field locations on the magnet allows stronger pull forces which allow changes in geometries in the design, allowing more impeller features on the magnet. Further, programmable multi-pole magnets allow a magnet construction with additional features that allow placement of attract and repel forces in close proximity to the coils. It is also possible to reduce blood sheer and resulting thrombus and ischemic events (primarily stroke). Finally, the impeller features can be incorporated into the magnet, thereby reducing part mass.

In an embodiment, a left ventricle assist device (LVAD) is disclosed, the LVAD having an inflow port, an outflow port, an impeller configured to pump blood from the inflow port to the outflow port so as to assist the left ventricle of a heart in pumping blood, and a motor driving the impeller, the motor is included having a stator element, a rotating disc element, wherein the rotating disc element includes programmable multi-pole magnets.

In an embodiment, the rotating disc element includes programmable multi-pole magnets configured for levitation.

In an embodiment, the rotating disc element includes programmable multi-pole magnets configured for application of rotational force to the rotating disc.

In an embodiment, the rotating disc element includes programmable multi-pole magnets configured for application of rotational force to the rotating disc.

In an embodiment, the rotating disc element includes programmable multi-pole magnets configured for application of rotational force to the rotating disc.

In an embodiment, the rotating disc includes programmable multi-pole magnets configured for application of rotational force and levitating force to the rotating disc.

In an embodiment, the rotating disc includes programmable multi-pole magnets configured for application of rotational force and levitating force to the rotating disc, wherein the same programmable multi-pole magnet is used for rotational force and levitating force.

In an embodiment, the rotating disc includes programmable multi-pole magnets configured for application of rotational force and levitating force to the rotating disc, wherein the programmable multi-pole magnets for the rotational force are located radially distinct from the programmable multi-pole magnets for levitation relative to the center of the disc.

In an embodiment, the rotating disc includes programmable multi-pole magnets configured for application of rotational force and levitating force to the rotating disc, wherein the programmable multi-pole magnets for the rotational force are located concentrically outside relative to the programmable multi-pole magnets for levitation.

In an embodiment, the rotating disc includes programmable multi-pole magnets configured for application of rotational force, wherein the programmable multi-pole magnets include alternating positive and negative magnetic regions.

In an embodiment, the rotating disc includes programmable multi-pole magnets configured for application of levitational force, wherein the programmable multi-pole magnets include continuous concentric regions of positive or negative magnetic polarity.

In an embodiment, the programmable multi-pole magnets include sintered NiCuNi material.

In an embodiment, the programmable multi-pole magnets include NdFeB material.

In an embodiment, the programmable multi-pole magnets include sintered SmCo material.

In an embodiment, the programmable multi-pole magnets include a PTFE material.

In an embodiment, the rotating disc includes an open interior for passage of blood, the open interior having an area at least 10 percent of the area within the circumference of the disc.

In an embodiment, the rotating disc includes an open interior for passage of blood, the open interior having an area at least 15 percent of the area within the circumference of the disc.

In an embodiment, the rotating disc includes an open interior for passage of blood, the open interior having an area at least 20 percent of the area within the circumference of the disc.

In an embodiment, the rotating disc includes an open interior for passage of blood, the open interior having an area at least 25 percent of the area within the circumference of the disc.

In an embodiment, the rotating disc includes an open interior for passage of blood, the open interior having an area at least 30 percent of the area within the circumference of the disc.

In an embodiment, the rotating disc includes an open interior for passage of blood, the open interior having an area at least 35 percent of the area within the circumference of the disc.

In an embodiment, the rotating disc includes an open interior for passage of blood, the open interior having an area at least 40 percent of the area within the circumference of the disc.

In an embodiment, the rotating disc includes an open interior for passage of blood, the open interior having an area at least 45 percent of the area within the circumference of the disc.

In an embodiment, the rotating disc includes an open interior for passage of blood, the open interior having an area at least 50 percent of the area within the circumference of the disc.

In an embodiment, the rotating disc includes an open interior for passage of blood, the open interior having an area at least 60 percent of the area within the circumference of the disc.

In an embodiment, the rotating disc includes an open interior for passage of blood, the open interior having an area at least 70 percent of the area within the circumference of the disc.

In an embodiment, wherein clearance above and below the rotating disc relative to adjacent components is less than 3 millimeters.

In an embodiment, wherein clearance above and below the rotating disc relative to adjacent components is less than 2 millimeters.

In an embodiment, wherein clearance above and below the rotating disc relative to adjacent components is less than 1 millimeter.

In an embodiment, wherein clearance above and below the rotating disc relative to adjacent components is less than 0.5 millimeters.

In an embodiment, the rotor has a thickness of less than 5 millimeters.

In an embodiment, the rotor has a thickness of less than 4 millimeters.

In an embodiment, the rotor has a thickness of less than 3 millimeters.

In an embodiment, the rotor has a thickness of less than 2 millimeters.

In an embodiment, the thickness of the programmable multi-pole magnets are less than 2 millimeters.

In an embodiment, the thickness of the programmable multi-pole magnet is less than 1 millimeter.

In an embodiment, the programmable multi-pole magnets are arranged in a radially symmetric pattern.

In an embodiment, the programmable multi-pole magnets cover at least 50 percent of the surface area of the rotor.

In an embodiment, the programmable multi-pole magnets cover at least 60 percent of the surface area of the rotor.

In an embodiment, the programmable multi-pole magnets cover at least 70 percent of the surface area of the rotor.

In an embodiment, the programmable multi-pole magnets cover at least 80 percent of the surface area of the rotor.

In an embodiment, the programmable multi-pole magnets cover at least 90 percent of the surface area of the rotor.

In an embodiment, the programmable multi-pole magnets cover at least 95 percent of the surface area of the rotor.

The present application is directed, in part, to LVADs that incorporate magnets having preferred design features, including discs having field emission structures comprising one or more electric or magnetic field sources having magnitudes, polarities, and positions corresponding to a desired spatial force function where a spatial force is created based upon the relative alignment of a field emission structure and a complementary field emission structure. Specific exemplary embodiments are described with magnetic field sources arranged in a ring structure. The ring structure may include one or more concentric rings of component magnets. Mechanical constraints may be employed to limit lateral motion.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The following drawings are illustrative of embodiments of the present invention and therefore do not limit the scope of the invention. The drawings are not to scale and are intended for use in conjunction with the explanations in the following detailed description. Like numerals may describe similar components in different views. Like numerals having different letter suffixes may represent different instances of similar components. The drawings illustrate generally, by way of example, but not by way of limitation, various embodiments discussed in the present document.

FIG. 1 is a simplified cross-sectional view of conventional left ventricle assist device.

FIG. 2 is a simplified cross-sectional view of a conventional left ventricle assist device.

FIG. 3 is a cross-sectional schematic view of elements of left ventricle assist device in accordance with various embodiments herein.

FIG. 4 is a perspective view of magnetic disc in accordance with various embodiments herein.

FIG. 5 is a top plan view of magnetic disc in accordance with various embodiments herein.

FIG. 6 is a top plan view of a magnetic disc according to various embodiments.

FIG. 7 is a top plan view of magnetic disc in accordance with various embodiments herein.

FIG. 8 is a top plan view of magnetic disc in accordance with various embodiments herein.

FIG. 9 is a top plan view of magnetic disc in accordance with various embodiments herein.

FIG. 10 is a top plan view of magnetic disc in accordance with various embodiments herein.

FIG. 11 is a top plan view of magnetic disc in accordance with various embodiments herein.

FIG. 12 is a top plan view of magnetic disc during manufacturing in accordance with various embodiments herein.

FIG. 13 is a cross-sectional schematic view of elements of left ventricle assist device in accordance with various embodiments herein.

FIG. 14 cross-sectional schematic view of elements of left ventricle assist device in accordance with various embodiments herein.

FIG. 15 are illustrations showing coil forces applied during operation of an LVAD in accordance with various embodiments herein.

FIG. 16 is a perspective view of left ventricle assist device in accordance with various embodiments herein.

FIG. 17 is an exploded perspective view of left ventricle assist device in accordance with various embodiments herein.

FIG. 18 is a perspective view of the impeller for a left ventricle assist device in accordance with various embodiments herein.

FIG. 19 is a perspective view of portions of a magnetic disc in accordance with various embodiments herein.

FIG. 20 is a perspective view of example coils from a magnetic disc in accordance with various embodiments herein.

FIG. 21A is a top plan view of a magnetic disc.

FIG. 21B is a top plan view of the magnetic disc during the CNC-controlled selective pole-reversal magnetization process.

FIG. 22A is a perspective view of the magnetic disc.

FIG. 22B is a schematic edge view of the magnetic disc.

FIG. 22C a side view of the magnetic disc and the one or more coils.

FIG. 23 is a schematic top view of the magnetic disk and the one or more coils relative to each other.

FIG. 24 is a schematic diagram of a left ventricle assist device.

FIG. 25 is a schematic perspective view of the outer radial edge region of the left ventricle assist device.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

In an embodiment, a left ventricle assist device (LVAD), the LVAD is included having an inflow port, an outflow port, an impeller configured to pump blood from the inflow port to the outflow port so as to assist the left ventricle of a heart in pumping blood, and a motor driving the impeller, the motor is included having a stator element, a rotating disc element, wherein the rotating disc element includes programmable multi-pole magnets.

In an embodiment, the rotating disc element includes programmable multi-pole magnets configured for levitation. In an embodiment, the rotating disc element includes programmable multi-pole magnets configured for application of rotational force to the rotating disc. In an embodiment, the rotating disc element includes programmable multi-pole magnets configured for application of rotational force to the rotating disc. In an embodiment, the rotating disc element includes programmable multi-pole magnets configured for application of rotational force to the rotating disc. In an embodiment, the rotating disc includes programmable multi-pole magnets configured for application of rotational force and levitating force to the rotating disc.

In an embodiment, the rotating disc includes programmable multi-pole magnets configured for application of rotational force and levitating force to the rotating disc, wherein the same programmable multi-pole magnets are used for rotational force and levitating force. In an embodiment, the rotating disc includes programmable multi-pole magnets configured for application of rotational force and levitating force to the rotating disc, wherein the programmable multi-pole magnets for the rotational force are located radially distinct from the programmable multi-pole magnets for levitation relative to the center of the disc.

In an embodiment, the rotating disc includes programmable multi-pole magnets configured for application of rotational force and levitating force to the rotating disc, wherein the programmable multi-pole magnets for the rotational force are located concentrically outside relative to the programmable multi-pole magnets for levitation.

In an embodiment, the rotating disc includes programmable multi-pole magnets configured for application of rotational force, wherein the programmable multi-pole magnets include alternating positive and negative magnetic regions. In an embodiment, the rotating disc includes programmable multi-pole magnets configured for application of levitational force, wherein the programmable multi-pole magnets include continuous concentric regions of positive or negative magnetic polarity.

In an embodiment, the programmable multi-pole magnets include sintered NiCuNi material. In an embodiment, the programmable multi-pole magnets include NdFEB material. In an embodiment, the programmable multi-pole magnets include sintered SmCo material. In an embodiment, the programmable multi-pole magnets include a PTFE material.

In an embodiment, the rotating disc includes an open interior for passage of blood, the open interior having an area at least 10 percent of the area within the circumference of the disc. In an embodiment, the rotating disc includes an open interior for passage of blood, the open interior having an area at least 15 percent of the area within the circumference of the disc. In an embodiment, the rotating disc includes an open interior for passage of blood, the open interior having an area at least 20 percent of the area within the circumference of the disc. In an embodiment, the rotating disc includes an open interior for passage of blood, the open interior having an area at least 25 percent of the area within the circumference of the disc. In an embodiment, the rotating disc includes an open interior for passage of blood, the open interior having an area at least 30 percent of the area within the circumference of the disc. In an embodiment, the rotating disc includes an open interior for passage of blood, the open interior having an area at least 35 percent of the area within the circumference of the disc. In an embodiment, the rotating disc includes an open interior for passage of blood, the open interior having an area at least 40 percent of the area within the circumference of the disc. In an embodiment, the rotating disc includes an open interior for passage of blood, the open interior having an area at least 45 percent of the area within the circumference of the disc. In an embodiment, the rotating disc includes an open interior for passage of blood, the open interior having an area at least 50 percent of the area within the circumference of the disc. In an embodiment, the rotating disc includes an open interior for passage of blood, the open interior having an area at least 60 percent of the area within the circumference of the disc. In an embodiment, the rotating disc includes an open interior for passage of blood, the open interior having an area at least 70 percent of the area within the circumference of the disc.

In an embodiment, wherein clearance above and below the rotating disc relative to adjacent components is less than 3 millimeters. In an embodiment, wherein clearance above and below the rotating disc relative to adjacent components is less than 2 millimeters. In an embodiment, wherein clearance above and below the rotating disc relative to adjacent components is less than 1 millimeter. In an embodiment, wherein clearance above and below the rotating disc relative to adjacent components is less than 0.5 millimeters.

In an embodiment, the rotor has a thickness of less than 5 millimeters. In an embodiment, the rotor has a thickness of less than 4 millimeters. In an embodiment, the rotor has a thickness of less than 3 millimeters. In an embodiment, the rotor has a thickness of less than 2 millimeters. In an embodiment, the thickness of the programmable multi-pole magnets are less than 2 millimeters. In an embodiment, the thickness of the programmable multi-pole magnets are less than 1 millimeter.

In an embodiment, the thickness of the programmable multi-pole magnets are arranged in a radially symmetric pattern.

In an embodiment, the programmable multi-pole magnets cover at least 50 percent of the surface area of the rotor. In an embodiment, the programmable multi-pole magnets cover at least 60 percent of the surface area of the rotor. In an embodiment, the programmable multi-pole magnets cover at least 70 percent of the surface area of the rotor. In an embodiment, the programmable multi-pole magnets cover at least 80 percent of the surface area of the rotor. In an embodiment, the programmable multi-pole magnets cover at least 90 percent of the surface area of the rotor. In an embodiment, the programmable multi-pole magnets cover at least 95 percent of the surface area of the rotor.

The present design allows for a reduction of mass by: (a) dramatic reduction of mass of the permanent rare earth; (b) reduction of size of windings, coils; and (c) the ability to make general reductions of all materials

The present design allows, in some embodiments, for a reduction in power consumption, allowing greater battery life and reduction in size of external power management systems

The present design allows, in some implementations, for greater proximities of coils to specific field locations on the magnet allows stronger pull forces which allow changes in geometries in the design, allowing more impeller features on the magnet

The present design allows, in some embodiments, for use of programmable multi-pole magnets that allow a magnet construction with additional features that will allow us to put the attract and repel forces in close proximity to the coils.

The present design allows, in some embodiments, for a reduction of blood sheer and resulting thrombus and ischemic events (primarily stroke).

The present design allows, in dome embodiments, for an impeller/magnet to become an assembly with greatly reduced part mass, allowing the assembly to be levitated in another axis such that the assembly is in levitation when the patient is in a horizontal (as when sleeping).

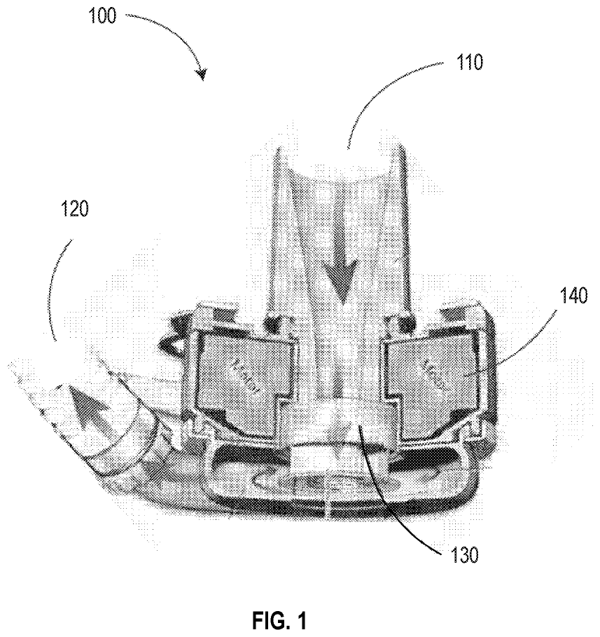

FIG. 1 is a simplified cross-sectional view of conventional left ventricle assist device 100, including an inlet 110 for receiving blood and outlet 120 for the blood. A motor assembly 140 is shown, along with a rotating disc and impeller assembly 130.

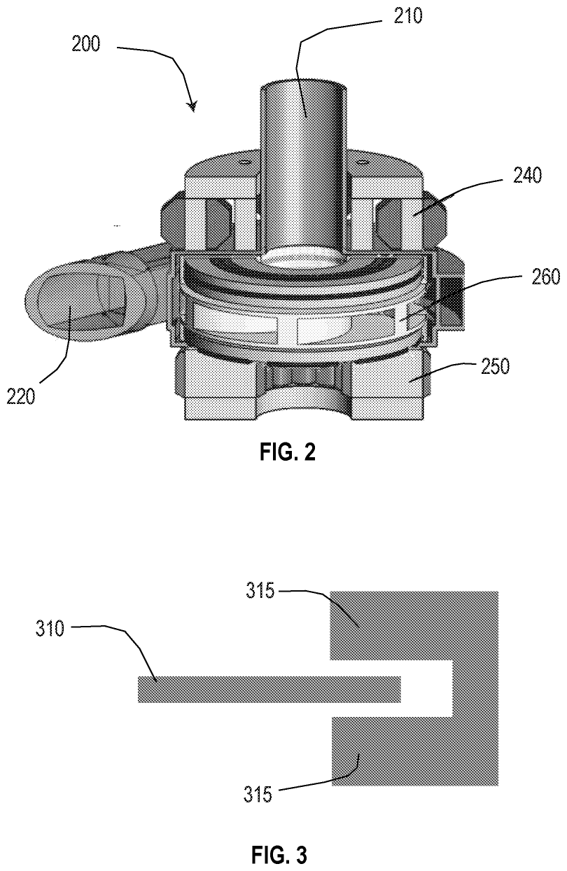

FIG. 2 is a simplified cross-sectional view of a conventional left ventricle assist device 200, including an inlet 210 for receiving blood and an outlet 220 for the blood. Also shown is a magnetic bearing stator 240, a magnetic stator 250, and a levitated impeller 260.

FIG. 3 is a cross-sectional schematic view of elements of left ventricle assist device in accordance with various embodiments herein, here showing levitation magnets 315 and rotating disc 310. In typical use there will be levitating magnets that perform a bearing function (allowing the disc to rotate with reduced friction), plus electric coils (not shown) that can engage alternating magnetic areas on the disc to cause it rotate (thereby providing the force to engage an impeller or other device to pressurize blood and pump it through the LVAD).

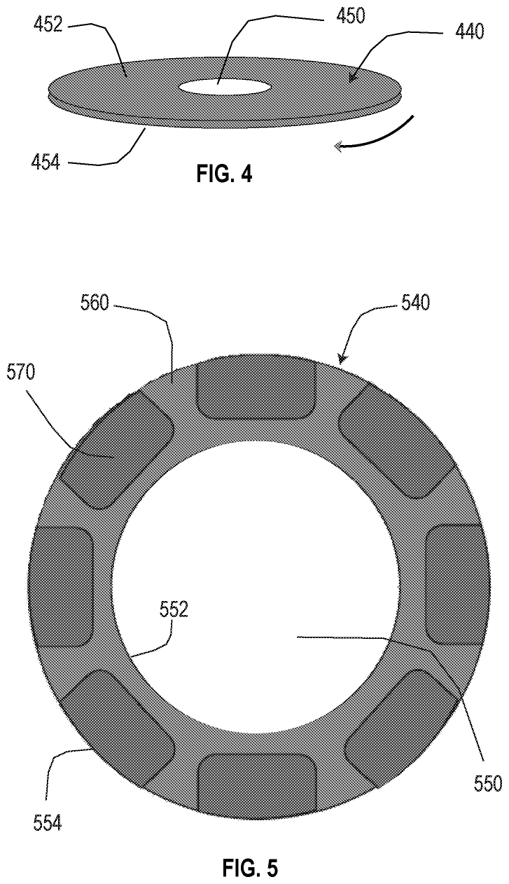

FIG. 4 is a perspective view of magnetic disc 440 in accordance with various embodiments herein, including an interior opening 450 (through which blood flows), and a top face 452 and bottom face 454. Top face 452 and bottom face 454 are magnetized using programmable multi-pole magnets process to provide a custom, durable magnetic disc with positive/negative (north/south) regions for engaging coils to drive rotation of the disc and (optionally) to provide a levitating force.

FIG. 5 is a top plan view of magnetic disc 540 in accordance with various embodiments herein, the magnetic disc 540 including an interior opening 550, the magnetic disc 540 interior opening 550 having an inner edge 552 and an outer edge 554. The magnetic disc 540 includes first area 560 having one magnetic polarity (such as “north” or “positive”) and second areas 570 having an opposite magnetic polarity (such as “south” or negative)”. In this embodiment the first area 560 extends around the entire magnetic disk 540, with eight distinct regions forming the second areas 570 having opposite magnetic polarity to the first area 560.

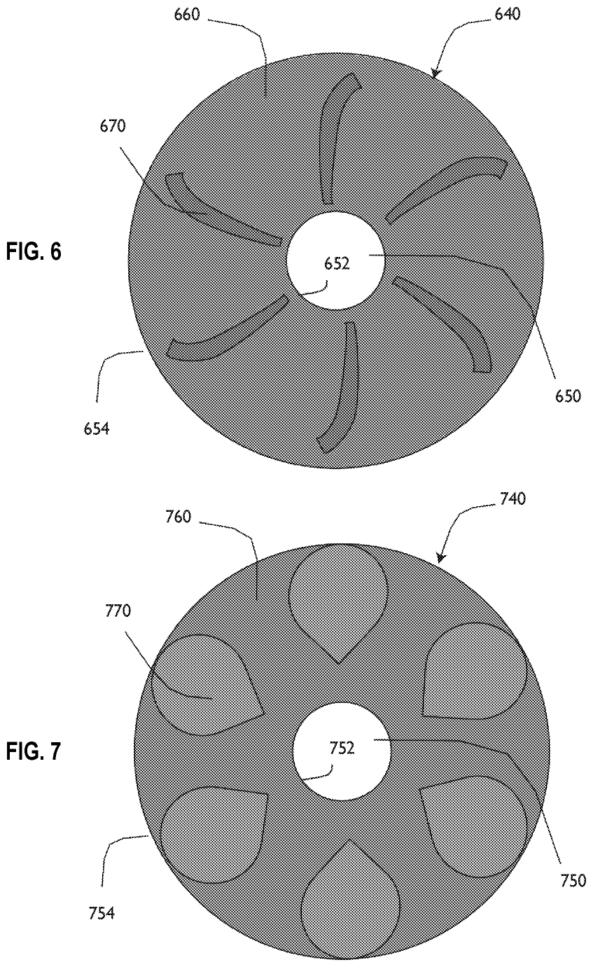

FIG. 6 is a top plan view of magnetic disc 640 in accordance with various embodiments herein, the magnetic disc 640 including an interior opening 650, the magnetic disc 640 interior opening 650 having an inner edge 652 and an outer edge 654. The magnetic disc 640 includes first area 660 having one magnetic polarity (such as “north” or “positive”) and second areas 670 having an opposite magnetic polarity (such as “south” or negative)”. In this embodiment the first area 660 extends around the entire magnetic disk 640, with six distinct regions forming the second areas 670 having opposite magnetic polarity to the first area 660. This embodiment also has a smaller interior opening 650 than the interior opening 550 of the magnetic disc 540.

FIG. 7 is a top plan view of magnetic disc 740 in accordance with various embodiments herein, the magnetic disc 740 including an interior opening 750, the magnetic disc 740 interior opening 750 having an inner edge 752 and an outer edge 754. The magnetic disc 740 includes first area 760 having one magnetic polarity (such as “north” or “positive”) and second areas 770 having an opposite magnetic polarity (such as “south” or negative)”. In this embodiment the first area 760 extends around the entire magnetic disk 740, with six distinct “teardrop” regions forming the second areas 770 having opposite magnetic polarity to the first area 760.

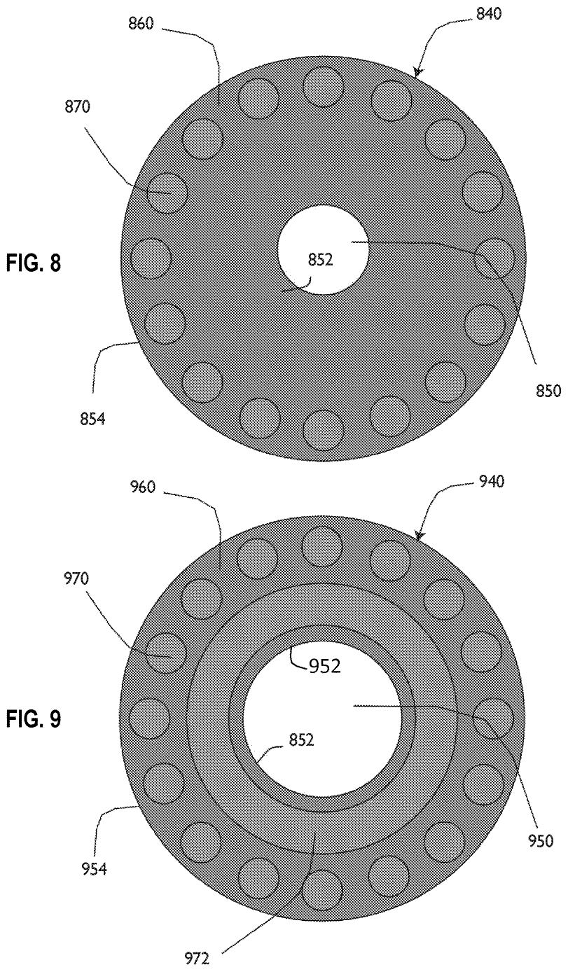

FIG. 8 is a top plan view of magnetic disc 840 in accordance with various embodiments herein, the magnetic disc 840 including an interior opening 850, the magnetic disc 840 interior opening 850 having an inner edge 852 and an outer edge 854. The magnetic disc 840 includes first area 860 having one magnetic polarity (such as “north” or “positive”) and second areas 870 having an opposite magnetic polarity (such as “south” or negative)”. In this embodiment the first area 860 extends around the entire magnetic disk 840, with sixteen distinct regions forming the second areas 870 having opposite magnetic polarity to the first area 860. The second areas 870 are positioned close to the outer edge 854 of the magnetic disc 840 so as to engage coils within an LVAD housing to impel rotation of the disc. The interior opening 850 is shown relatively small, but can be formed to be much larger (similar to FIG. 5).

FIG. 9 is a top plan view of magnetic disc 940 in accordance with various embodiments herein, the magnetic disc 940 including an interior opening 950, the magnetic disc 940 interior opening 950 having an inner edge 952 and an outer edge 954. The magnetic disc 940 includes first area 960 having one magnetic polarity (such as “north” or “positive”) and second areas 970 having an opposite magnetic polarity (such as “south” or negative)”. In this embodiment the first area 960 extends around the entire magnetic disk 940, with sixteen distinct regions forming the second areas 970 having opposite magnetic polarity to the first area 960. In addition region 972 is magnetized w/the same polarity as second areas 970, and can be used

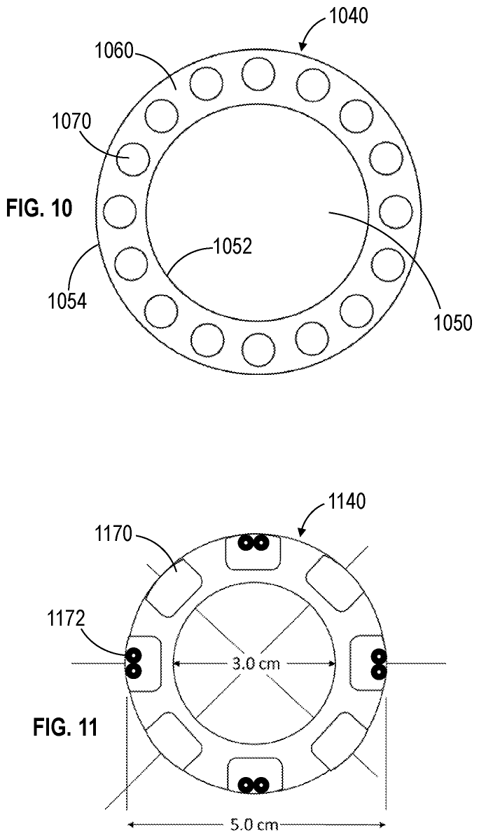

FIG. 10 is a top plan view of magnetic disc 1040 in accordance with various embodiments herein, the magnetic disc 1040 including an interior opening 1050, the magnetic disc 1040 interior opening 1050 having an inner edge 1052 and an outer edge 1054. The magnetic disc 1040 includes first area 1060 having one magnetic polarity (such as “north” or “positive”) and second areas 1070 having an opposite magnetic polarity (such as “south” or negative)”. In this embodiment the first area 1060 extends around the entire magnetic disc 1040, with sixteen distinct regions forming the second areas 1070 having opposite magnetic polarity to the first area 1060.

FIG. 11 is a top plan view of magnetic disc 1140 in accordance with various embodiments herein, showing area 1170 with a magnetic field, as well as sensors 1172.

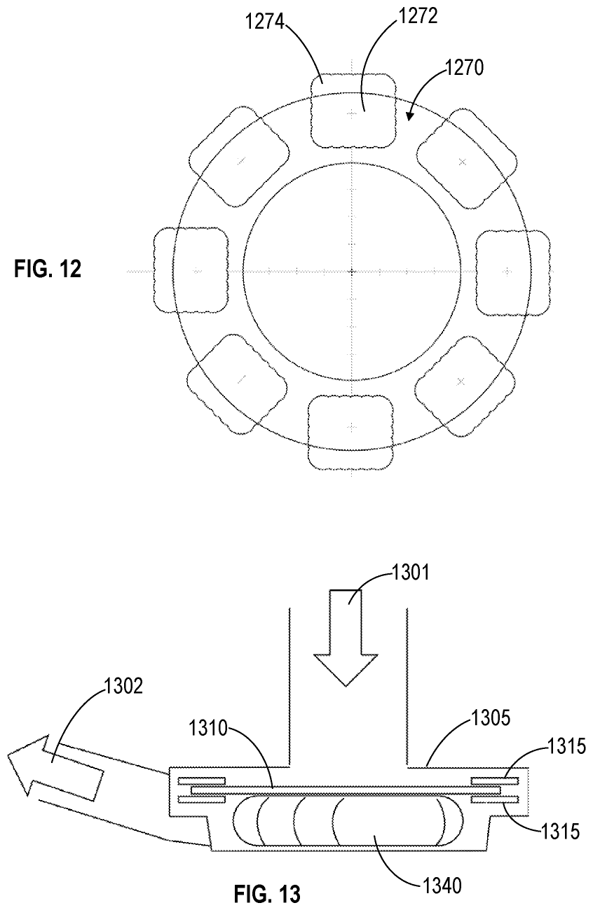

FIG. 12 is a top plan view of magnetic disc 1270 during manufacturing in accordance with various embodiments herein. FIG. 12 shows the disc 1270 with a magnetic area 1272 that will remain with the disc after manufacturing, along with an area 1274 that is removed during processing.

FIG. 13 is a cross-sectional schematic view of elements of left ventricle assist device in accordance with various embodiments herein, including an inlet 1301, an outlet 1302, a housing 1305, magnets 1315, disc 1310, and impeller assembly 1340.

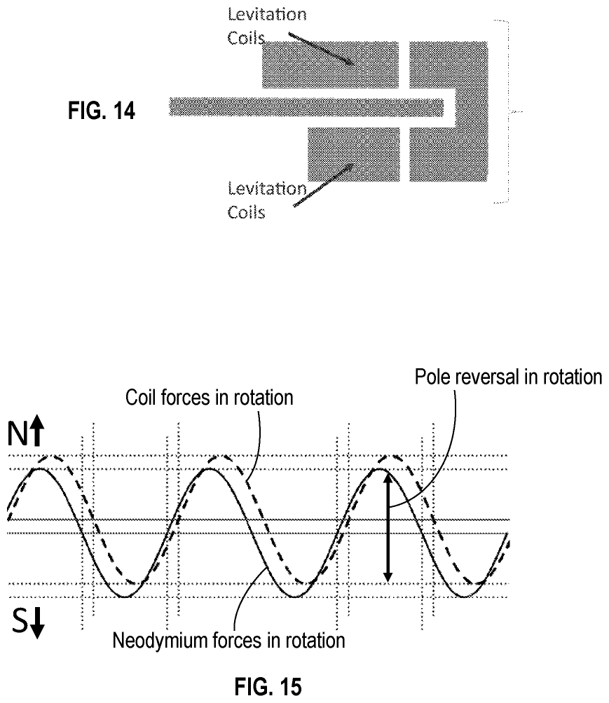

FIG. 14 cross-sectional schematic view of elements of left ventricle assist device in accordance with various embodiments herein, showing how levitation coils are positioned above and below a disc, and where drive coils are positioned on the outer circumference of the disc. Coils serve to both rotate and levitate the magnet/impeller assembly. They move the magnet in rotation by creating a temporary magnetic field of opposite polarities, above and below, to attract the fixed fields of the magnet. In addition, the strength of the field of a given coil is dynamically varied to continually position (“levitate”) the magnet/impeller assembly. This allows the magnet to levitate vertically, horizontally, and to be positioned dynamically in two ways: (a) centered in the device; (b) equidistant between the coils (“floating ”).

In an optimized version, the magnet is reduced in mass as much as possible by opening the center, since the primary function is at the outer area. In an optimized version, the magnet is affixed to the impeller and the impeller is the primary, mechanical structure.

Coils serve to both rotate and levitate the magnet/impeller assembly. They move the magnet in rotation by creating a temporary magnetic field of opposite polarities, above and below, to attract the fixed fields of the magnet.

In addition, the strength of the field of a given coil is dynamically varied to continually position (“levitate”) the magnet/impeller assembly. This allows the magnet to levitate vertically, horizontally, and to be positioned dynamically in two ways: (a) centered in the device; (b) equidistant between the coils (“floating”).

FIG. 15 are illustrations showing coil forces applied during operation of an LVAD in accordance with various embodiments herein, showing how the forces change, including pole reversal, to rotate the disc.

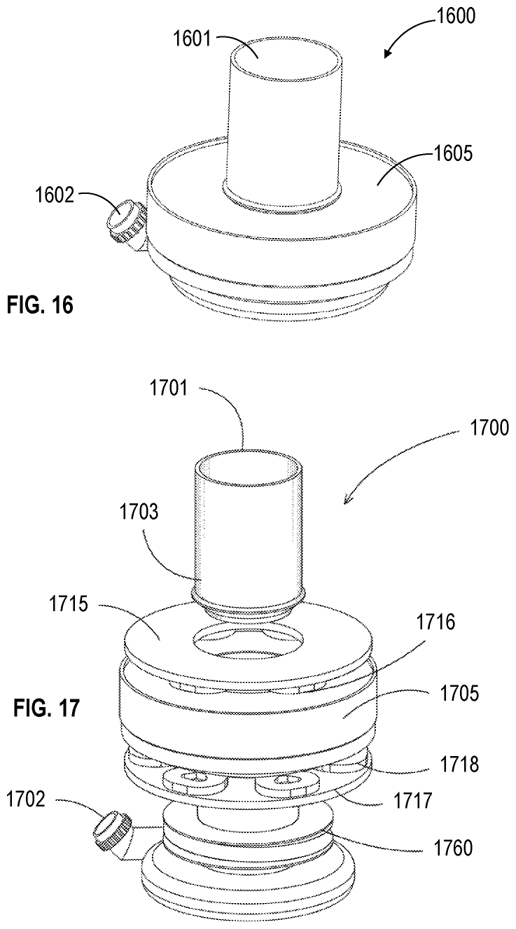

FIG. 16 is a perspective view of left ventricle assist device 1600 in accordance with various embodiments herein, showing an inlet 1601, outlet 1602 and body 1605.

FIG. 17 is an exploded perspective view of left ventricle assist device 1700 in accordance with various embodiments herein, including an inlet 1701, outlet 1702, first disc 1715 and second disc 1717, a body housing 1705, coils 1716 and 1718, plus an impeller assembly 1760.

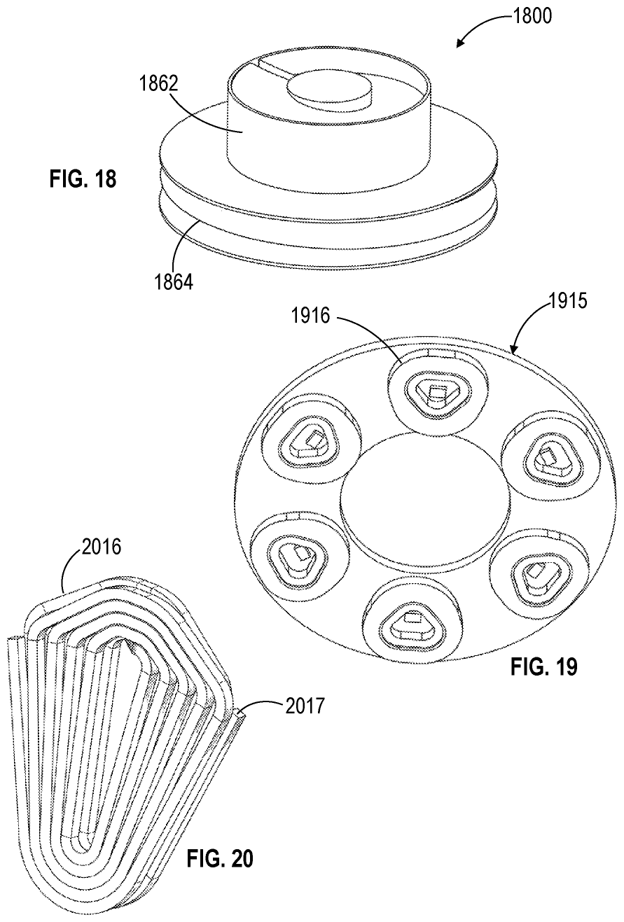

FIG. 18 is a perspective view of an impeller assembly 1800 for a left ventricle assist device in accordance with various embodiments herein, including a first portion 1862 and second portion 1864 with a greater diameter than the first portion. Generally it is desirable to reduce blood sheer by having a larger opening and larger impeller, with more impelling surface and reduce “edge length”.

The impeller can be made of UHMW-PE with coating of PTFE. An alternative is PEX. Two parts join together to capture the plastic parts to the magnet. UHMW has high molecular weight and can stand very high number of cycles. Alternatively titanium may be used.

FIG. 19 is a perspective view of portions of a magnetic disc 1915 in accordance with various embodiments herein, showing coils 1916. In practice, the coils 1916 can be flush with the surrounding surface of the disc.

FIG. 20 is a perspective view of example coils from a magnetic disc in accordance with various embodiments therein. Each coil 2016 can be formed from a wire 2017 having rectangular or square cross-sectional profile.

The rotating discs described herein may be formed using the systems and processes termed “POLYMAGNETS™” by Correlated Magnetics Research, LLC, including as described in U.S. Pat. Nos. 9,406,424; 8,570,129; 8,174,347; 9,082,539; 9,367,783; 8,844,412; 10,173,292; 8,872,608; 7,755,462; 9,105,384; 8,917,715; 8,937,521; and 10,194,246.

Axial flux motor configurations can achieve substantial improvements in rotational torque generation relative to conventional motor configurations by applying magnetic force at the maximum radial distance from the center of rotation. In an axial flux configuration, a rotating disc element can be configured to rotate about a central axis, with magnets disposed at the outer radial edge of the rotating disc element. One or more coils can be positioned in a circumferential array surrounding the outer radial edge of the rotating disc element, with each coil arranged such that its active magnetic face can be directed radially inward toward the outer circumferential surface of the magnetic disc. This topology can leverage the mechanical principle that rotational torque is the product of applied force and the perpendicular distance from the axis of rotation, such that positioning the coils at the outer radial edge can maximize the moment arm and the rotational torque generated per unit of magnetic force applied. As a result, the rotational torque produced per unit of electrical input power can be substantially increased relative to conventional motor configurations in which coils are positioned centrally beneath or above the disc face. Axial flux configurations can achieve power-to-mass ratios of orders of magnitude greater than conventional motor configurations.

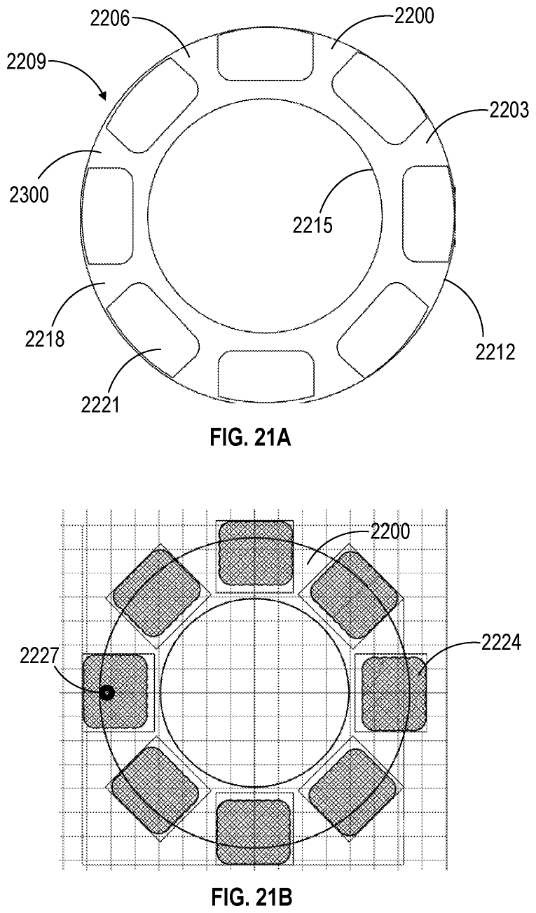

The axial flux configurations are applied to a Left Ventricular Assist Device (LVAD). Construction of the LVAD and the method of operation is illustrated in FIGS. 21 through 25 and discussed below. The LVAD illustrated in FIGS. 21-25 may optionally incorporate one or more features, components, or configurations of the LVAD embodiments described herein with respect to FIG. 120. The LVAD described below can provide an improvement in power-to-mass ratio of approximately six times compared to conventional LVAD motor configurations. This improvement can enable the use of a larger impeller operating at a lower rotational speed for a given blood flow output, which can reduce the acceleration forces applied to blood cells and thereby reduce hemolysis, thrombus formation, and ischemic events including stroke.

FIG. 21A is a top plan view of a magnetic disc 2200. The magnetic disk 2200 can include a programmable multi-pole magnet (e.g., POLYMAGNET™) after completion of a CNC-controlled selective pole-reversal magnetization process, illustrating the resulting polar region pattern on the face of the magnetic disc 2200. FIG. 21B is a top plan view of the magnetic disc 2200 during the CNC-controlled selective pole-reversal magnetization process, illustrating the CNC magnetization machine pathway used to define and create the alternating polarity regions on the magnetic disc 2200. FIG. 21A and FIG. 21B together illustrate the manufacturing process by which a single piece of neodymium magnetic material can be selectively magnetized to produce a magnetic disc 2200 having permanent alternating north and south polar regions along its outer radial edge without requiring mechanical assembly of multiple discrete magnet pieces.

The magnetic disc 2200 can be formed from a single piece of neodymium magnetic material 2203, in an embodiment comprising plated neodymium, having a disc geometry (shown in FIG. 4) with a first face 2206 (e.g., top face 452 of FIG. 4) and a second face 2209 opposing the first face 2206 (e.g., the bottom face 454 of FIG. 4). The single piece of neodymium magnetic material 2203 can be initially magnetized with a uniform first polarity orientation perpendicular to the first face 2206, such that the entirety of the first face 2206 presents a uniform north polarity and the opposing second face 2209 presents a uniform south polarity prior to the selective pole-reversal process.

The magnetic disc 2200 can have an outer diameter 2212 (e.g., approximately 5.0 centimeters) and an internal diameter 2215 (e.g., approximately 3.0 centimeters). As illustrated in FIG. 21A, the first face 2206 of the magnetic disc 2200 can include a first polarity region 2218 extending across a majority of the first face 2206 and presenting a first magnetic polarity. One or more second polarity regions 2221 can be disposed within the first polarity region 2218 at circumferentially spaced apart locations along the outer radial edge of the magnetic disc 2200, the one or more second polarity regions 2221 presenting a second magnetic polarity opposite to the first magnetic polarity. In the illustrated embodiment, the one or more second polarity regions 2221 can comprise eight discrete regions of reversed polarity, defining eight alternating south polar regions interspersed with eight north polar regions along the outer radial edge of the magnetic disc 2200. The number of alternating polar regions can be varied between 6 and 16 by modifying the programming of the CNC magnetization machine, without requiring physical tooling changes or mechanical assembly of multiple discrete magnet pieces.

As illustrated in FIG. 21B, a CNC magnetization machine pathway 2224 can define the positions, extents, and geometries of the one or more second polarity regions 2221 to be created on the first face 2206 of the magnetic disc 2200. The CNC magnetization machine pathway 2224 can represent the programmed traverse path of a focused reverse magnetic field applicator across the surface of the single piece of neodymium magnetic material 2203, configured to permanently reverse the polarity of discrete regions as defined by the programmed polar pattern. The CNC-controlled manufacturing process can enable any desired polar pattern to be produced by modifying the CNC program, without requiring physical tooling changes or mechanical assembly of multiple discrete magnet pieces.

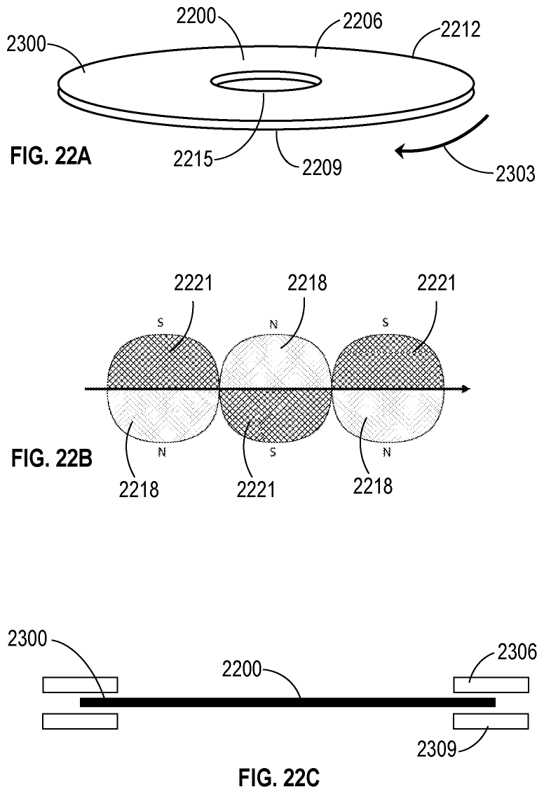

The CNC magnetization machine pathway 2224 can further be configured to create at least one position sensing feature 2227 on the first face 2206 of the magnetic disc 2200 by defining a geometrically distinct magnetic region whose polarity or boundary geometry can be detectable by a sensor co-located with the one or more coils 2109, shown in FIG. 22B (e.g., coils 1716 or 1718 of FIG. 17) of the left ventricle assist device, enabling a controller to determine the rotational position and pole orientation of the magnetic disc 2200 in real time during operation.

FIG. 22A illustrates a perspective view of the magnetic disc 2200. The magnetic disc 2200 can have a first face 2206 and a second face 2209 extending between the outer diameter 2212 and an inner diameter 2215. The magnetic disc 2200 can be substantially flat.

FIG. 22B is a schematic edge view of the magnetic disc 2200 illustrating the alternating north and south polar regions disposed along the outer circumferential edges of the first face 2206 and the second face 2209 of the magnetic disc 2200. The first polarity region 2218 and the one or more second polarity regions 2221 can alternate in a circumferential pattern along the outer circumferential surface 2300 of the magnetic disc 2200, presenting a repeating sequence of north and south magnetic poles as viewed from the outer radial edge of the magnetic disc 2200. In the illustrated embodiment, eight alternating polar regions can be disposed along the outer circumferential surface 2300, defining eight pole transitions around the circumference of the magnetic disc 2200.

FIG. 22C illustrates a side view of the magnetic disc 2200 and the one or more coils 2109. The one or more coils 2109 can include upper coils 2306 positioned across the first face 2206 of the magnetic disc 2200 and lower coils 2309 positioned across the second face 2209 of the magnetic disc 2200. The upper coils 2306 and the lower coils 2309 can cooperate to engage the alternating polar regions of the outer circumferential surface 2300 from both above and below to maximize the rotational torque and levitation force applied to the magnetic disc 2200.

FIG. 23 is a schematic top view of the magnetic disk 2200 and the one or more coils 2109 relative to each other, illustrating the attract force 2312 generated between each coil of the one or more coils 2109 and the alternating polar regions of the outer circumferential surface 2300 of the magnetic disc 2200, showing how the coil fields interact with the fixed magnetic fields of the magnetic disc 2200 to apply rotational torque and levitation force. FIG. 23 illustrates the mechanism by which the one or more coils 2109 can drive a rotation of the magnetic disc 2200 and simultaneously generate levitation force through differential variation of coil pulse intensity.

As the magnetic disc 2200 rotates in a rotation direction 2303, the alternating north and south polar regions of the outer circumferential surface 2300 can move relative to the fixed positions of the one or more coils 2109, creating a changing magnetic field at each coil position that can be used to drive and sustain rotation of the magnetic disc 2200. The one or more coils 2109 can be positioned across the outer circumferential surface 2300 of the magnetic disc 2200 in a circumferential array surrounding the outer radial edge of the magnetic disc 2200, with coils positioned both above and below the outer radial edge of the magnetic disc. Each coil of the one or more coils 2109 can be configured to rapidly alternate between north and south polarity in synchronization with the rotation of the magnetic disc 2200, generating a temporary magnetic field that can attract the alternating polar regions of the outer circumferential surface 2300 and pull the magnetic disc 2200 into continuous rotation. The coils can drive rotation by generating temporary magnetic fields of alternating polarity that can attract the fixed alternating polarity regions of the outer radial edge of the disc, causing the disc to rotate, and the same coils can simultaneously perform levitation by differential variation of the pulse intensity applied to individual coils within the circumferential array.

The intensity of the attract force 2312 can be dynamically varied by adjusting the pulse intensity of individual coils of the one or more coils 2109, enabling differential coil power control. During times when the patient is in a vertical standing position, the field strength of the upper coils 2306 can be increased relative to the lower coils 2309, generating a net upward levitation force on the magnetic disc 2200 that counteracts gravitational loading. During times when the patient is in a horizontal reclined position, the upper coils 2306 and the lower coils 2309 can receive equal pulse power, and coils positioned at the apex of the arc of rotation of the magnetic disc 2200 can pulse together at higher power to generate a centering levitation force that maintains the magnetic disc 2200 in a centered and levitated position within the housing. In this manner, the one or more coils 2109 can simultaneously perform both rotation and multi-axis levitation of the magnetic disc 2200.

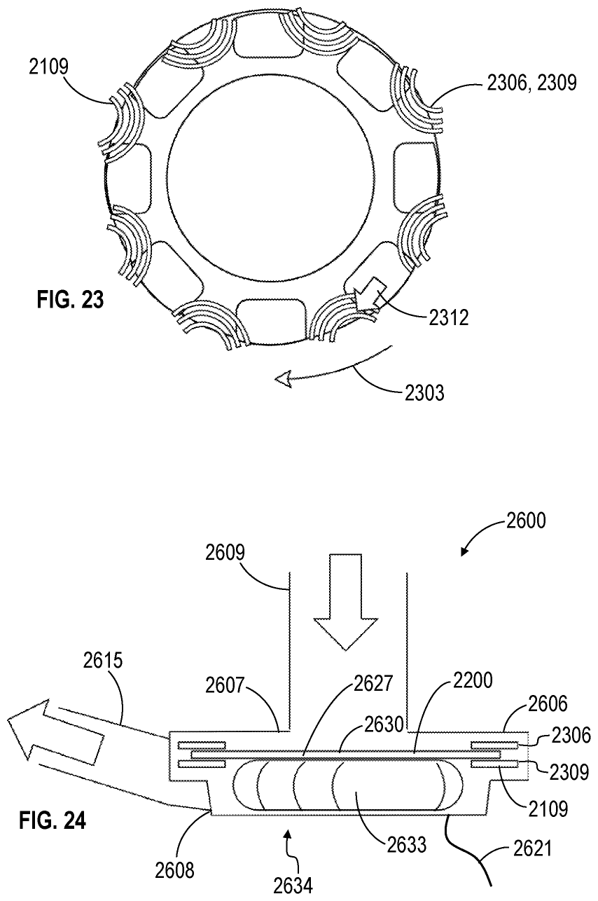

FIG. 24 is a schematic diagram of a left ventricle assist device 2600 according to an example configuration of the current disclosure. FIG. 24 illustrates the key structural features of the axial flux motor configuration and magnetic disc assembly and identifying the design improvements achieved relative to conventional left ventricle assist devices. The left ventricle assist device 2600 can include a housing 2606 configured to enclose the operative components of the device and to provide structural support for the one or more coils 2109 and the magnetic disc 2200. The housing 2606 can have a significantly reduced overall height relative to conventional left ventricle assist devices, the reduction in height being achieved primarily through reduction in the thickness of the pump body enabled by the axial flux motor configuration.

An inflow cannula 2609 can be disposed at a first end 2607 of the housing 2606 and can be configured for surgical attachment to the apex of the left ventricle of the heart. The inflow cannula 2609 can be similar in size to that of conventional left ventricle assist devices while the overall pump body thickness can be substantially reduced. An outflow tube 2615 can be disposed at a second end 2608 of the housing 2606 and can be configured to direct pressurized blood toward the ascending aorta.

A pump cable 2621 can extend from the housing 2606 and can be configured to transmit electrical power and control signals between the left ventricle assist device 2600 and an external controller and power source.

The magnetic disc 2200 can be positioned within the housing 2606 as a rotor magnet 2627 configured to rotate about a central axis of the left ventricle assist device 2600. The rotor magnet 2627 can have a thickness in the range of 2.0 millimeters to 3.5 millimeters, representing a significant reduction in magnet mass relative to conventional LVAD rotor magnets. The magnetic disc 2200 can include an open interior 2630 extending through the center of the rotor magnet 2627, the open interior 2630 being substantially enlarged relative to conventional left ventricle assist devices to reduce obstruction to blood flow and reduce acceleration forces applied to blood cells. An impeller assembly 2633 can be coupled to the magnetic disc 2200 to form a combined rotor and impeller assembly 2634. Reduced mass of the combined rotor and impeller assembly 2634 can facilitate magnetic levitation by the one or more coils 2109.

In some example configurations, the impeller assembly 2633 can be formed from ultra-high molecular weight polyethylene (UHMW-PE) with a coating of polytetrafluoroethylene (PTFE) applied to blood-contacting surfaces, UHMW-PE being selected for its high molecular weight, which provides resistance to wear and the ability to withstand a very high number of mechanical cycles as required for a chronically implanted blood pump, and the PTFE coating reducing surface friction and thrombogenicity on blood-contacting surfaces. In other example configurations, the impeller assembly 2633 can be formed from cross-linked polyethylene (PEX).

The impeller assembly 2633 can be enlarged relative to the impeller of conventional left ventricle assist devices, enabling operation at a lower rotational speed for a given blood flow rate, thereby reducing acceleration forces applied to blood cells and reducing hemolysis, thrombus formation, and ischemic events including stroke.

The one or more coils 2109 can be positioned in an axial flux configuration surrounding the outer radial edge of the magnetic disc 2200, with the upper coils 2306 positioned above the plane of the magnetic disc 2200 and the lower coils 2309 positioned below the plane of the magnetic disc 2200, enabling multi-axis levitation and continuous positioning of the combined rotor and impeller assembly 2634 within the housing 2606 without requiring separate mechanical bearing structures, in contrast to conventional left ventricle assist devices which employ dedicated magnetic bearing stators separate from the drive coil assembly.

The controller can receive signals from position sensors and field sensors co-located with the one or more coils 2109 and can use those signals to determine the current position and pole orientation of the magnetic disk 2200, dynamically maintaining levitation of the magnetic disk 2200 and impeller assembly in any patient orientation without requiring separate mechanical bearing structures.

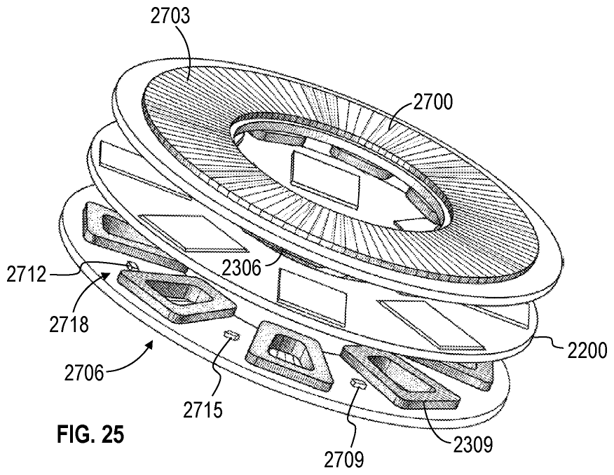

FIG. 25 is a schematic perspective view of the outer radial edge region of the left ventricle assist device. A field amplification backing member 2700 can be positioned behind each coil of the one or more coils 2109 on the side of each coil facing away from the magnetic disc 2200. The field amplification backing member 2700 can be a thin sheet of a high-permeability magnetic alloy configured to redirect and concentrate the magnetic flux generated by each coil toward the outer circumferential surface of the magnetic disc 2200, thereby amplifying the effective magnetic field at the surface of the outer radial edge of the magnetic disc 2200. In an example configuration, the field amplification backing member 2700 can include Vicalloy, which is a cobalt-vanadium-iron alloy. In another example configuration, the field amplification backing member 2700 can include silicon steel. The field amplification backing member 2700 can have a thickness of less than 2 millimeters and can be configured as a thin foil sheet, enabling field amplification with minimal addition to the overall mass or dimensions of the left ventricle assist device.

A first field amplification backing member 2703 can be positioned behind each of the upper coils 2306 on the side facing away from the magnetic disc 2200, and a second field amplification backing member 2706 can be positioned behind each of the lower coils 2309 on the side facing away from the magnetic disc 2200. The first field amplification backing member 2703 and the second field amplification backing member 2706 can cooperate to amplify the magnetic fields of the upper coils 2306 and the lower coils 2309 respectively in the direction of the outer circumferential surface of the magnetic disc 2200, increasing the efficiency of both rotational torque generation and levitation force generation without requiring increased electrical power input or increased coil size.

As illustrated in FIG. 25, one or more sensors 2709 can be co-located with the one or more coils 2109 at circumferentially spaced positions around the outer radial edge of the magnetic disc 2200. The one or more sensors 2709 can include position sensors 2712 configured to detect the rotational position of the magnetic disc 2200 in real time and field sensors 2715 configured to detect the pole orientation of the alternating polar regions of the magnetic disc 2200 as they pass each sensor position during rotation.

The one or more sensors 2709 can be arranged in sensor sets 2718, each sensor set 2718 including at least one position sensor 2712 and at least one field sensor 2715, configured to provide both positional and polarity information to a controller of the left ventricle assist device. The controller can use the signals from the one or more sensors 2709 to time the pulses applied to individual coils of the one or more coils 2109 for rotational drive and to adjust the differential pulse intensity among individual coils of the one or more coils 2109 to maintain the levitated position of the magnetic disc 2200 and the impeller assembly 2633 mechanically integrated therewith within the housing 2606 of the left ventricle assist device in response to detected patient orientation and real-time position data.

### ADDITIONAL NOTES AND EXAMPLES

Example 1 is a left ventricle assist device (LVAD), the LVAD comprising: a housing; an inflow port; an outflow port; an impeller configured to pump blood from the inflow port to the outflow port so as to assist the left ventricle of a heart in pumping blood; a magnetic disc mechanically coupled to the impeller, the magnetic disc comprising a programmable multi-pole magnet having alternating magnetic polarity regions disposed along an outer radial edge of the magnetic disc; and one or more coils affixed to the housing in an axial flux configuration, wherein the one or more coils are positioned proximate to the outer radial edge of the magnetic disc such that the coils engage the alternating magnetic polarity regions along the outer radial edge to apply rotational torque to the magnetic disc.

In Example 2, the subject matter of Example 1 optionally includes wherein the one or more coils are arranged in a circumferential array surrounding the outer radial edge of the magnetic disc, with coils positioned both above and below a plane of the magnetic disc, wherein each coil of the one or more coils is positioned with its central axis oriented radially with respect to the magnetic disc such that magnetic flux generated by each coil is directed radially inward toward the outer radial edge of the magnetic disc, and wherein the one or more coils are configured to both apply rotational torque to the magnetic disc and to dynamically levitate the magnetic disc through differential variation of pulse intensity among individual coils of the one or more coils.

In Example 3, the subject matter of Example 2 optionally includes wherein the differential variation of pulse intensity is controlled by a controller responsive to at least one position sensor and at least one field sensor co-located with the one or more coils, the controller configured to adjust pulse intensity of individual coils to maintain a centered position of the magnetic disc within the housing.

In Example 4, the subject matter of any one or more of Examples 2-3 optionally include wherein in a first patient orientation corresponding to a vertical standing position, coils positioned above the magnetic disc receive a higher pulse power than coils positioned below the magnetic disc, thereby generating an upward levitation force on the magnetic disc.

In Example 5, the subject matter of any one or more of Examples 2-4 optionally include wherein in a second patient orientation corresponding to a horizontal reclined position, coils positioned above and below the magnetic disc at an apex of an arc of rotation of the disc pulse together at a higher power level than remaining coils, thereby generating a centering levitation force on the magnetic disc.

In Example 6, the subject matter of any one or more of Examples 1-5 optionally include a field amplification backing member positioned behind each coil of the one or more coils on a side of each coil facing away from the magnetic disc, wherein the field amplification backing member comprises a material selected from the group including a magnetically soft iron-vanadium-cobalt alloy (commercially available under the trade name Vicalloy™) and silicon steel, and wherein the field amplification backing member is a thin sheet having a thickness of less than 2 millimeters.

In Example 7, the subject matter of Example 6 optionally includes wherein the field amplification backing member is configured to amplify the magnetic field generated by each coil in a direction toward the outer radial edge of the magnetic disc.

In Example 8, the subject matter of any one or more of Examples 1-7 optionally include wherein the magnetic disc is formed from a single piece of neodymium material having selectively reversed polarity regions created by a CNC-controlled magnetization process, the selectively reversed polarity regions defining the alternating magnetic polarity regions along the outer radial edge.

In Example 9, the subject matter of any one or more of Examples 1-8 optionally include millimeters.

In Example 10, the subject matter of any one or more of Examples 1-19 optionally include wherein the impeller is directly integrated with the magnetic disc to form a single assembly configured for levitation by the one or more coils.

In Example 11, the subject matter of Example 10 optionally includes wherein the impeller comprises ultra-high molecular weight polyethylene with a polytetrafluoroethylene coating.

In Example 12, the subject matter of any one or more of Examples 10-11 optionally include wherein the impeller comprises cross-linked polyethylene.

In Example 13, the subject matter of any one or more of Examples 1-12 optionally include wherein wires forming each coil of the one or more coils have a rectangular or square cross-sectional profile.

Example 14 is a left ventricle assist device (LVAD), the LVAD comprising: a housing; an inflow port; an outflow port; a magnetic disc having alternating magnetic polarity regions; an impeller mechanically integrated with the magnetic disc to form a combined rotor and impeller assembly configured to pump blood from the inflow port to the outflow port; one or more coils affixed to the housing and positioned proximate to an outer radial edge of the magnetic disc in an axial flux configuration; a field amplification backing member positioned behind each coil on a side facing away from the magnetic disc, the field amplification backing member comprising iron-vanadium-cobalt alloy or silicon steel; and a controller configured to vary pulse intensity of individual coils of the one or more coils to perform both rotation of the magnetic disc and multi-axis levitation of the combined rotor and impeller assembly.

In Example 15, the subject matter of Example 14 optionally includes wherein the controller is configured to determine a patient orientation and to adjust differential pulse intensity among the one or more coils in response to the determined patient orientation to maintain levitation of the combined rotor and impeller assembly, and wherein the controller is further configured to receive signals from one or more position sensors and field sensors co-located with the one or more coils and to use those signals to determine position and pole orientation of the magnetic disc in real time.

In Example 16, the subject matter of any one or more of Examples 14-15 optionally include wherein the one or more coils are arranged in a circumferential array comprising coils positioned above and below the outer radial edge of the magnetic disc.

In Example 17, the subject matter of any one or more of Examples 14-16 optionally include percent of the area within a circumference of the magnetic disc.

Example 18 is a method of operating a left ventricle assist device (LVAD) comprising a magnetic disc having alternating magnetic polarity regions along its outer radial edge and one or more coils positioned proximate to the outer radial edge in an axial flux configuration, the method comprising: detecting a patient orientation using at least one sensor; in response to detecting a first patient orientation corresponding to a vertical standing position, supplying a higher pulse power to coils positioned above the magnetic disc than to coils positioned below the magnetic disc to generate an upward levitation force on the magnetic disc; and in response to detecting a second patient orientation corresponding to a horizontal reclined position, supplying a higher pulse power to coils positioned at an apex of an arc of rotation of the magnetic disc than to remaining coils to generate a centering levitation force on the magnetic disc.

In Example 19, the subject matter of Example 18 optionally includes simultaneously pulsing the one or more coils with alternating polarity to apply rotational torque to the magnetic disc while performing levitation.

In Example 20, the subject matter of any one or more of Examples 18-19 optionally include receiving real-time position and field sensor signals from sensors co-located with the one or more coils and using those signals to adjust differential coil pulse intensity to maintain a centered position of the magnetic disc within a housing.

Each of these non-limiting examples can stand on its own or can be combined in any permutation or combination with any one or more of the other examples.

The above detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

What is claimed is:

1. A left ventricle assist device (LVAD), the LVAD comprising:
a housing;
an inflow port;
an outflow port;
an impeller configured to pump blood from the inflow port to the outflow port so as to assist the left ventricle of a heart in pumping blood;
a magnetic disc mechanically coupled to the impeller, the magnetic disc comprising a programmable multi-pole magnet having alternating magnetic polarity regions disposed along an outer radial edge of the magnetic disc; and
a plurality of coils affixed to the housing in an axial flux configuration, wherein the plurality of coils are positioned proximate to the outer radial edge of the magnetic disc such that the coils engage the alternating magnetic polarity regions along the outer radial edge to apply rotational torque to the magnetic disc.

2. The LVAD of claim 1, wherein the plurality of coils are arranged in a circumferential array surrounding the outer radial edge of the magnetic disc, with coils positioned both above and below a plane of the magnetic disc,
wherein each coil of the plurality of coils is positioned with its central axis oriented radially with respect to the magnetic disc such that magnetic flux generated by each coil is directed radially inward toward the outer radial edge of the magnetic disc, and
wherein the plurality of coils are configured to both apply rotational torque to the magnetic disc and to dynamically levitate the magnetic disc through differential variation of pulse intensity among individual coils of the plurality of coils.

3. The LVAD of claim 2, wherein the differential variation of pulse intensity is controlled by a controller responsive to at least one position sensor and at least one field sensor co-located with the plurality of coils, the controller configured to adjust pulse intensity of individual coils to maintain a centered position of the magnetic disc within the housing.

4. The LVAD of claim 2, wherein in a first patient orientation corresponding to a vertical standing position, coils positioned above the magnetic disc receive a higher pulse power than coils positioned below the magnetic disc, thereby generating an upward levitation force on the magnetic disc.

5. The LVAD of claim 2, wherein in a second patient orientation corresponding to a horizontal reclined position, coils positioned above and below the magnetic disc at an apex of an arc of rotation of the disc pulse together at a higher power level than remaining coils, thereby generating a centering levitation force on the magnetic disc.

6. The LVAD of claim 1, further comprising a field amplification backing member positioned behind each coil of the plurality of coils on a side of each coil facing away from the magnetic disc,
wherein the field amplification backing member comprises a material selected from the group including iron-vanadium-cobalt alloy and silicon steel, and
wherein the field amplification backing member is a thin sheet having a thickness of less than 2 millimeters.

7. The LVAD of claim 6, wherein the field amplification backing member is configured to amplify the magnetic field generated by each coil in a direction toward the outer radial edge of the magnetic disc.

8. The LVAD of claim 1, wherein the magnetic disc is formed from a single piece of neodymium material having selectively reversed polarity regions created by a CNC-controlled magnetization process, the selectively reversed polarity regions defining the alternating magnetic polarity regions along the outer radial edge.

9. The LVAD of claim 1, wherein the magnetic disc has a thickness in a range of 2.0 millimeters to 3.5 millimeters.

10. The LVAD of claim 1, wherein the impeller is directly integrated with the magnetic disc to form a single assembly configured for levitation by the plurality of coils.

11. The LVAD of claim 10, wherein the impeller comprises ultra-high molecular weight polyethylene with a polytetrafluoroethylene coating.

12. The LVAD of claim 10, wherein the impeller comprises cross-linked polyethylene.

13. The LVAD of claim 1, wherein wires forming each coil of the plurality of coils have a rectangular or square cross-sectional profile.

14. A left ventricle assist device (LVAD), the LVAD comprising:
a housing;
an inflow port;
an outflow port;
a magnetic disc having alternating magnetic polarity regions;
an impeller mechanically integrated with the magnetic disc to form a combined rotor and impeller assembly configured to pump blood from the inflow port to the outflow port;
a plurality of coils affixed to the housing and positioned proximate to an outer radial edge of the magnetic disc in an axial flux configuration;
a field amplification backing member positioned behind each coil on a side facing away from the magnetic disc, the field amplification backing member comprising iron-vanadium-cobalt alloy or silicon steel; and
a controller configured to vary pulse intensity of individual coils of the plurality of coils to perform both rotation of the magnetic disc and multi-axis levitation of the combined rotor and impeller assembly.

15. The LVAD of claim 14, wherein the controller is configured to determine a patient orientation and to adjust differential pulse intensity among the plurality of coils in response to the determined patient orientation to maintain levitation of the combined rotor and impeller assembly, and
wherein the controller is further configured to receive signals from a plurality of position sensors and field sensors co-located with the plurality of coils and to use those signals to determine position and pole orientation of the magnetic disc in real time.

16. The LVAD of claim 14, wherein the plurality of coils are arranged in a circumferential array comprising coils positioned above and below the outer radial edge of the magnetic disc.

17. The LVAD of claim 14, wherein the magnetic disc includes an open interior for passage of blood, the open interior having an area of at least 30 percent of the area within a circumference of the magnetic disc.

18. A method of operating a left ventricle assist device (LVAD) comprising a magnetic disc having alternating magnetic polarity regions along its outer radial edge and a plurality of coils positioned proximate to the outer radial edge in an axial flux configuration, the method comprising:
detecting a patient orientation using at least one sensor;
in response to detecting a first patient orientation corresponding to a vertical standing position, supplying a higher pulse power to coils positioned above the magnetic disc than to coils positioned below the magnetic disc to generate an upward levitation force on the magnetic disc; and
in response to detecting a second patient orientation corresponding to a horizontal reclined position, supplying a higher pulse power to coils positioned at an apex of an arc of rotation of the magnetic disc than to remaining coils to generate a centering levitation force on the magnetic disc.

19. The method of claim 18, further comprising simultaneously pulsing the plurality of coils with alternating polarity to apply rotational torque to the magnetic disc while performing levitation.

20. The method of claim 18, further comprising receiving real-time position and field sensor signals from sensors co-located with the plurality of coils and using those signals to adjust differential coil pulse intensity to maintain a centered position of the magnetic disc within a housing.
